(19) United States
(12) Patent Application Publication
(10) Pub. No.: US 2026/0016466 A1
(43) Pub. Date: Jan. 15, 2026

(54) TEST PIECE FOR MEASURING ALBUMIN AND METHOD FOR MEASURING ALBUMIN

(71) Applicant: FUJIFILM Corporation, Tokyo (JP)

(72) Inventors: Yoshihiro ABURAYA, Ashigarakami-gun (JP); Masaki MURAKAMI, Ashigarakami-gun (JP); Manabu KOBAYASHI, Ashigarakami-gun (JP)

(73) Assignee: FUJIFILM Corporation, Tokyo (JP)

(21) Appl. No.: 19/334,036

(22) Filed: Sep. 19, 2025

Related U.S. Application Data
(63) Continuation of application No. PCT/JP2024/010646, filed on Mar. 19, 2024.

(30) Foreign Application Priority Data
Mar. 20, 2023 (JP) 2023-044052

(51) Int. Cl. G01N 33/52; G01N 33/68; G01N 33/84

(52) U.S. Cl. CPC G01N 33/526; G01N 33/6839; G01N 33/84; G01N 2333/76

(57) ABSTRACT

An object of the present invention is to provide a test piece for measuring albumin, which is not affected by globulin or coexisting substances in serum, have high measurement accuracy, and have good repeatability, and to provide a method for measuring albumin.

According to the present invention, there is provided a test piece for measuring albumin, including a support and a reagent holding layer provided over the support, in which the reagent holding layer contains a protein denaturant, an SH reagent, bromocresol purple, and a nonionic surfactant, a content of the bromocresol purple in the reagent holding layer is 0.78 g/m² to 3.81 g/m², and a ratio of a content of the nonionic surfactant to the content of the bromocresol purple is 0.01 to 0.3.

### CROSS-REFERENCE TO RELATED APPLICATIONS

This application is a Continuation of PCT International Application No. PCT/JP2024/010646 filed on Mar. 19, 2024, which claims priority under 35 U.S.C § 119 (a) to Japanese Patent Application No. 2023-044052 filed on Mar. 20, 2023. Each of the above application(s) is hereby expressly incorporated by reference, in its entirety, into the present application.

### BACKGROUND OF THE INVENTION

### 1. Field of the Invention

The present invention relates to a test piece for measuring albumin and a method for measuring albumin, which use bromocresol purple.

### 2. Description of the Related Art

As a test strip for measuring serum albumin, a test piece using a bromocresol green method (BCG method) is widespread. Since the BCG method is affected by globulin and develops color in the presence of globulin, a test piece using a bromocresol purple method (BCP method) that is not affected by globulin and has high specificity to albumin is desired.

Although a test piece shown in JP2003-270249A is disclosed as an albumin test piece using the BCP method, the BCP method is known to have a disadvantage of being susceptible to interference by coexisting substances in serum. On the other hand, JP1998-232233A (JP-H10-232233A) discloses that albumin is measured by a solution method using a BCP measurement reagent that is not affected by coexisting substances in serum, as a reagent for measuring serum albumin. Furthermore, JP2022-085876A discloses a test piece in which a protein denaturant and an SH reagent are contained to change reduced albumin to oxidized albumin, which is then reacted with BCP, as a test piece that overcomes the disadvantage of being susceptible to interference by the coexisting substance in serum.

### SUMMARY OF THE INVENTION

On the other hand, in a case of analyzing a component in serum, such as albumin, in addition to the performance of not being affected by other substances, it is desired to have high repeatability of measurement, that is, the performance of showing the same measurement value even in a case of repeated measurement. An object of the present invention is to provide a test piece for measuring albumin, which is not affected by globulin or coexisting substances in serum and has good repeatability, and to provide a method for measuring albumin.

As a result of diligent studies to achieve the above-described object, the present inventors have found that, in a test piece for measuring albumin, which includes a reagent holding layer containing a protein denaturant, an SH reagent, bromocresol purple, and a nonionic surfactant, by setting a content of bromocresol purple within a predetermined range and setting a ratio of a content of the nonionic surfactant to the content of bromocresol purple within a predetermined range, high repeatability in measurement values is achieved. The present invention has been completed based on the above findings. According to the present invention, the following inventions are provided.

<1> A test piece for measuring albumin, comprising: a support; and a reagent holding layer provided over the support, in which the reagent holding layer contains a protein denaturant, an SH reagent, bromocresol purple, and a nonionic surfactant, a content of the bromocresol purple in the reagent holding layer is 0.78 g/m² to 3.81 g/m², and a ratio of a content of the nonionic surfactant to the content of the bromocresol purple is 0.01 to 0.3.

<2> The test piece for measuring albumin according to <1>, in which the content of the bromocresol purple in the reagent holding layer is 0.78 g/m² to 2.21 g/m², and the ratio of the content of the nonionic surfactant to the content of the bromocresol purple is 0.01 to 0.2.

<3> The test piece for measuring albumin according to <1> or <2>, in which the reagent holding layer contains a water-soluble polymer.

<4> The test piece for measuring albumin according to <3>, in which the water-soluble polymer in the reagent holding layer is polyvinylpyrrolidone.

<5> The test piece for measuring albumin according to any one of <1> to <4>, further comprising an interlayer between the support and the reagent holding layer.

<6> The test piece for measuring albumin according to <5>, in which the interlayer contains a water-soluble polymer, and a content of the water-soluble polymer in the interlayer is 10 to 100 g/m².

<7> The test piece for measuring albumin according to any one of <1> to <6>, in which the nonionic surfactant is a nonionic surfactant having a polyoxyethylene group.

<8> The test piece for measuring albumin according to any one of <1> to <7>, in which the nonionic surfactant is one or more nonionic surfactants selected from the group consisting of polyoxyethylene lauryl ether, polyoxyethylene oleyl ether, polyoxyethylene sorbitan monolaurate, polyoxyethylene sorbitan monooleate, polyoxyethylene tert-octylphenyl ether, and polyoxyethylene isooctylphenyl ether.

<9> The test piece for measuring albumin according to any one of <1> to <8>, in which the protein denaturant is sodium dodecyl sulfate.

<10> The test piece for measuring albumin according to any one of <1> to <9>, in which the SH reagent is 5,5′-dithiobis(2-nitrobenzoic acid).

<11> The test piece for measuring albumin according to any one of <1> to <10>, in which pH of a liquid of the reagent holding layer is 4.5 to 5.5.

<12> A method for measuring albumin, comprising: spotting an albumin-containing sample on the test piece for measuring albumin according to any one of <1> to <11>; and measuring color development in the reagent holding layer.

According to the test piece for measuring albumin and the method for measuring albumin according to the embodiment of the present invention, it is possible to perform measurement with good repeatability.

### DESCRIPTION OF THE PREFERRED EMBODIMENTS

Hereinafter, the present invention is specifically described.

In the present specification, “to” shows a range including numerical values described before and after “to” as a minimum value and a maximum value, respectively.

The present invention relates to a test piece for measuring albumin, comprising: a support; and a reagent holding layer provided over the support, in which the reagent holding layer contains a protein denaturant, an SH reagent, bromocresol purple, and a nonionic surfactant, a content of the bromocresol purple in the reagent holding layer is 0.78 g/m² to 3.81 g/m², and a ratio of a content of the nonionic surfactant to the content of the bromocresol purple is 0.01 to 0.3.

In the present invention, the repeatability of the measured value can be increased by satisfying that the content of the bromocresol purple in the reagent holding layer is 0.78 g/m² to 3.81 g/m², and the ratio of the content of the nonionic surfactant to the content of the bromocresol purple is 0.01 to 0.3.

By setting the ratio of the content of the nonionic surfactant to the content of the bromocresol purple to 0.3 or less, denaturation of albumin can be suppressed, whereby a decrease in reactivity between albumin and BCP can be prevented. By setting the ratio of the content of the nonionic surfactant to the content of the bromocresol purple to 0.01 or more, decrease in solubility of albumin can be suppressed, whereby a decrease in reactivity between albumin and BCP can be prevented.

As the protein denaturant, an anionic surfactant is preferable. In one or a plurality of embodiments, examples of the anionic surfactant include an alkyl sulfate surfactant, a polyoxyethylene alkylphenyl ether sulfate, a polyoxyethylene alkyl ether sulfate, an alkylbenzene sulfonate, and the like. In one or a plurality of embodiments, examples of the alkyl sulfate surfactant include sodium dodecyl sulfate (SDS), sodium cetyl sulfate, and the like. In one or a plurality of embodiments, examples of the polyoxyethylene alkylphenyl ether sulfate include sodium polyoxyethylene alkylphenyl ether sulfate, and the like.

In one or a plurality of embodiments, examples of the polyoxyethylene alkyl ether sulfate include sodium polyoxyethylene alkyl ether sulfate, triethanolamine polyoxyethylene alkyl ether sulfate, and the like. In one or a plurality of embodiments, examples of the alkylbenzenesulfonate include sodium laurylbenzenesulfonate, sodium cetylbenzenesulfonate, and the like. Among these, sodium dodecyl sulfate is particularly preferable.

The amount of the protein denaturant used in the reagent holding layer is preferably 0.075 g/m² to 7.5 g/m².

Examples of the SH reagent include disulfide compounds such as 5,5′-dithiobis(2-nitrobenzoic acid) (DTNB), 2,2′-dithiobis(5-nitropyridine) (DTNP), 2,2′-dithiodipyridine (2-PDS), 4,4′-dithiodipyridine (4-PDS), 4,4′-dithiobis(phenylazide) (DTBPA), and oxidized glutathione; oxidizing agents such as iodine, ferricyanides, iodosobenzoic acid, iodate salts, chlorate salts, mercury, and zinc; alkylating agents such iodoacetic acid, chloroacetic acid, iodoacetamide, and chloroacetophenone; maleimide; maleimide derivatives such as N-methylmaleimide, N-ethylmaleimide, and N,N′-p-phenylenedimalemide; thiophthalimide; and the like. Among these, for example, a disulfide compound such as DTNB, 2-PDS, or 4-PDS, maleimide; a maleimide derivative such as N-ethylmaleimide, or the like is particularly preferably used. DTNB is particularly preferable as the SH reagent. In addition, these may be used alone or in appropriate combinations.

The amount of the SH reagent used in the reagent holding layer is preferably 0.01 g/m² to 1 g/m².

As the bromocresol purple (BCP), a commercially available product can be used. BCP is used as a color developing agent for albumin measurement.

In the present invention, the content of bromocresol purple in the reagent holding layer is 0.78 g/m² to 3.81 g/m², and is preferably 0.78 g/m² to 2.21 g/m².

As the nonionic surfactant, a nonionic surfactant having a polyoxyethylene group is preferable. As the nonionic surfactant, more preferably, one or more nonionic surfactants selected from the group consisting of polyoxyethylene lauryl ether, polyoxyethylene oleyl ether, polyoxyethylene sorbitan monolaurate, polyoxyethylene sorbitan monooleate, polyoxyethylene tert-octylphenyl ether, and polyoxyethylene isooctylphenyl ether, can be used.

Examples of the polyoxyethylene oleyl ether include polyoxyethylene (20) oleyl ether, polyoxyethylene (23) lauryl ether (Brij35), polyoxyethylene (50) oleyl ether, and the like.

As the nonionic surfactant, Triton X-100, Tween 20, Tween 80, Triton X-405, Emalgen 108, Emalgen 120, Emalgen 150, Emalgen 430, or the like can also be used.

The coating amount of the nonionic surfactant in the test piece for measuring albumin is preferably 0.01 g/m² to 0.4 g/m².

In the present invention, the ratio of the content of the nonionic surfactant to the content of the bromocresol purple is 0.01 to 0.3 and preferably 0.01 to 0.2.

The test piece for measuring albumin according to the embodiment of the present invention includes a support and a reagent holding layer provided over the support.

Preferably, the test piece for measuring albumin according to the embodiment of the present invention further includes an interlayer between the support and the reagent holding layer. In this case, the test piece for measuring albumin according to the embodiment of the present invention includes a support, an interlayer, and a reagent holding layer.

More preferably, the test piece for measuring albumin according to the embodiment of the present invention includes a support; an interlayer; and a spreading layer including a reagent holding layer.

The interlayer is preferably a layer (water absorption layer) composed of a water-soluble polymer that absorbs water and swells as a main component.

The spreading layer is a layer having an action (metering action) of spreading an aqueous liquid sample, which is supplied in spots onto the upper surface of the test piece for measuring albumin, in a lateral direction without substantially unevenly distributing the components contained in the aqueous liquid sample, and supplying the aqueous liquid sample to an interlayer containing a water-soluble polymer having a water absorbency at a ratio of a substantially constant capacity per unit area.

In addition, a layer such as an adhesive layer can also be provided between the support and the layer provided thereon and between the respective layers provided on the support.

In addition, the above-described interlayer and spreading layer may be provided as separate layers, but may also be provided as one layer by allowing the same layer to have the above-described two or more functions at the same time. That is, the interlayer and the spreading layer may be provided as the same layer.

As the support, a water-impermeable support is preferable. As the material of the water-impermeable support, a polymer such as polyethylene terephthalate, polycarbonate of bisphenol A, polystyrene, or cellulose ester (for example, cellulose diacetate, cellulose triacetate, cellulose acetate propionate, or the like) is preferable, and polyethylene terephthalate is particularly preferable. As the support, a smooth planar support that is transparent, for example, transmits electromagnetic radiation having a wavelength in a range of at least a part of a wavelength range of approximately 200 nm to approximately 900 nm, in a thickness range of approximately 50 μm to approximately 1 mm and preferably approximately 80 μm to approximately 300 μm can be used. A known undercoat or adhesive layer can be provided on the surface of the support to strengthen the adhesion to the interlayer.

The interlayer is preferably a layer containing a water-soluble polymer that can swell and absorb water by being brought into contact with water. The interlayer has an action of improving the development of the aqueous liquid sample in the spreading layer in a case where the water in the aqueous liquid sample spotted on the spreading layer reaches the upper surface of the interlayer during the analysis operation.

The content of the water-soluble polymer in the interlayer is preferably 10 to 100 g/m² and more preferably 20 to 70 g/m².

The water-soluble polymer used in the interlayer is preferably a non-proteinaceous water-soluble polymer having a property of swelling and absorbing water in a case of being in contact with water. Examples of the non-proteinaceous water-soluble polymer include acrylamide-based copolymers such as polyacrylamide, agarose, and acrylamide-N-vinylpyrrolidone copolymer described in JP1982-50660A (JP-S57-50660A), JP1983-77664A (JP-S58-77664A), and the like; methallyl alcohol-based copolymers such as a binary or ternary copolymer of methallyl alcohol with acrylamide or a derivative thereof, acrylic acid or a derivative thereof, methacrylic acid or a derivative thereof, or N-vinyl-2-pyrrolidone, described in JP1987-137564A (JP-S62-137564A) (the methallyl alcohol-based copolymer is crosslinkable, for example, an acrylamide-N-vinyl-2-pyrrolidone-methallyl alcohol ternary copolymer); and the like.

Among these non-proteinaceous water-soluble polymers, an acrylic amide-based copolymer such as an acrylamide-N-vinylpyrrolidone copolymer or a methallyl alcohol-containing copolymer such as an acrylamide-N-vinyl-2-pyrrolidone-methallyl alcohol ternary copolymer is preferable.

The interlayer can contain various components that do not adversely affect the capability of BCP. An example thereof is a nonionic surfactant. Specific examples of the nonionic surfactant include the same ones as the surfactant that can be contained in the reagent holding layer. By containing the nonionic surfactant in the interlayer, water in the aqueous liquid sample is likely to be substantially uniformly absorbed into the interlayer during the analysis operation, and the liquid contact with the spreading layer is rapidly and substantially uniform. In addition, the interlayer can contain a buffering agent described later.

The interlayer can contain a crosslinking agent (also referred to as a curing agent or a hardening agent). As the crosslinking agent, various inorganic and organic crosslinking agents well known in the field of organic polymer chemistry can be used. Examples of the organic crosslinking agent for polyvinyl alcohol include dimethyl urea, and examples of the organic crosslinking agent for a methallyl alcohol-containing polymer include formaldehyde. The content of the crosslinking agent in the interlayer can be selected depending on the coating amount and the degree of curing of the interlayer to be crosslinked and cured, but is generally in a range of approximately 50 mg/m² to approximately 5,000 mg/m² and preferably in a range of approximately 100 mg/m² to approximately 2,000 mg/m² in terms of the coating amount.

As the spread layer, for example, a woven spreading layer (for example, a plain woven fabric such as broadcloth or poplin) described in JP1980-164356A (JP-S55-164356A), JP1982-66359A (JP-S57-66359A), or the like; a knitted spreading layer (for example, a tricot knitted fabric, a double tricot knitted fabric, a Milanese knitted fabric, or the like) described in JP1985-222769A (JP-S60-222769A) or the like; a spreading layer consisting of wet-laid paper containing organic polymer fiber pulp as described in JP1982-148250A (JP-S57-148250A); a nonfibrous isotropic porous spreading layer such as a membrane filter (brush polymer layer) as described in JP1978-21677A (JP-S53-21677A) and U.S. Pat. No. 3,992,158A, or a continuous microporous porous layer in which polymer microbeads, glass microbeads, or diatomaceous earth are held by a water-soluble polymer binder; a nonfibrous isotropic porous spreading layer composed of a continuous microporous porous layer (three-dimensional lattice-like particulate structure layer) in which polymer microbeads are bond in point contact with each other by a polymer adhesive that does not swell or expand in water as described in JP1980-90859A (JP-S55-90859A); or the like can be used. As the spreading layer, a knitted spreading layer (for example, a tricot knitted fabric, a double tricot knitted fabric, a Milanese knitted fabric, or the like) is preferable.

The spreading layer includes a reagent holding layer containing a protein denaturant, an SH reagent, bromocresol purple, and a nonionic surfactant. The reagent holding layer can further include a pH adjuster and a water-soluble polymer.

Examples of the water-soluble polymer that can be included in the reagent holding layer include acrylic polymers (such as polyacrylamide), cellulose-based polymers (such as carboxymethyl cellulose and hydroxymethyl cellulose), and the like. As the water-soluble polymer, polyvinyl alcohol, polyvinyl ether, polyvinylpyrrolidone, or the like can be preferably used.

The coating amount of the water-soluble polymer in the reagent holding layer is in a range of approximately 0.1 g/m² to approximately 30 g/m² and preferably approximately 0.5 g/m² to approximately 20 g/m².

The pH of a liquid of the reagent holding layer is preferably 4.5 to 5.8, more preferably 4.6 to 5.7, and still more preferably 4.7 to 5.6. In the reaction between albumin and bromocresol purple, since the amount of reaction increases as the pH approaches 7, but the coloring of bromocresol purple itself (background) also increases, it is considered that the above range is preferable. The pH of a liquid of the reagent holding layer means the pH of the coating liquid used for forming the reagent holding layer.

As the pH adjuster for adjusting the pH of the reagent holding layer to the pH in the above-described range, succinic acid, disodium succinate, or sodium hydroxide is preferable, and succinic acid is more preferable.

The interlayer and the spreading layer can contain a buffering agent.

As the buffering agent, at least one organic acid selected from the group consisting of hydroxycarboxylic acid and dicarboxylic acid is used. Examples of the hydroxycarboxylic acid include malic acid, lactic acid, citric acid, and tartaric acid. Examples of the dicarboxylic acid include malonic acid, succinic acid, glutaric acid, adipic acid, pimelic acid, and 3,3-dimethylglutaric acid. Among the above, succinic acid is preferable.

The content of the buffering agent is in a range of approximately 30 milliequivalents to approximately 500 milliequivalents and preferably approximately 50 milliequivalents to approximately 300 milliequivalents per 1 m². It is preferable that the buffering agent is dispersed in the water-soluble polymer together with other reagents and dispersedly held in one or more layers of the interlayer, the reagent holding layer, and the spreading layer.

Examples of the method for including the reagent holding layer in the spreading layer include a method of substantially uniformly applying or spraying an aqueous solution, a water-organic solvent mixed solvent solution, or an organic solvent solution (examples of the organic solvent: aliphatic alcohols such as methanol, ethanol, and isopropyl alcohol; dialkyl ketones such as acetone and methyl ethyl ketone; dialkyl ethers such as dimethyl ether; aliphatic cyclic ethers such as tetrahydrofuran and dioxane; acetonitrile; hexane; β-methoxyethanol; ethylene glycol; and the like) containing a protein denaturant, an SH reagent, bromocresol purple, and a nonionic surfactant from above the spreading layer and drying by a known method; a method of immersing a material for a spreading layer in a solution containing a protein denaturant, an SH reagent, bromocresol purple, and a nonionic surfactant and laminating the material on an interlayer (water-soluble polymer binder layer) in a dry or semi-dry state to integrate the material; and the like.

The test piece for measuring albumin according to the embodiment of the present invention can be prepared by a method known to those skilled in the art. For example, a coating liquid prepared as an interlayer coating liquid is applied onto the support and dried to produce a dried film having a thickness of about 40 μm, and then a woven fabric of the spreading layer is bonded thereto. Thereafter, the coating liquid prepared as the reagent holding layer liquid is applied from the woven fabric side of the spreading layer and dried, whereby a test piece for measuring albumin can be prepared. It is preferable that the test piece for measuring albumin is cut into small pieces such as a square having one side of approximately 15 mm to approximately 30 mm or substantially the same size as the square, and used as a chemical analysis slide by being housed in a slide frame described in JP1982-28331B (JP-S57-28331B), JP1981-142454B (JP-S56-142454B), JP1982-63452A (JP-S57-63452A), JP1983-32350A (JP-S58-32350A), JP1983-501144A (JP-S58-501144A), from various viewpoints such as manufacturing, packaging, transportation, storage, and measurement operations. Depending on the intended use, the test piece can be used in a long tape shape by being housed in a cassette or a magazine, or can be used in a small piece shape by being attached to or housed in a card having an opening.

According to the present invention, there is provided a method for measuring albumin, comprising: spotting an albumin-containing sample on the test piece for measuring albumin according to the embodiment of the present invention; and measuring color development in the reagent holding layer.

For example, the content of albumin in the liquid sample can be determined by spotting an aqueous liquid sample such as whole blood, blood plasma, serum, lymph fluid, or urine, in a range of approximately 5 μL to approximately 30 μL and preferably approximately 8 μL to approximately 15 μL, on the spreading layer, incubating the aqueous liquid sample at a substantially constant temperature in a range of approximately 20° C. to approximately 40° C. and preferably at a substantially constant temperature in the vicinity of 37° C. for a range of approximately 1 minute to approximately 10 minutes and preferably approximately 2 minutes to approximately 7 minutes, and measured, by reflection photometry, the detectable change such as the color change or the color development in the test piece for measuring albumin from the support side, according to the principle of the colorimetry. In the present invention, the optical density of the spreading layer is measured by reflection photometry using light having an absorption maximum wavelength of albumin-BCP binding or a wavelength in the vicinity thereof, and the albumin content in the liquid sample can be determined according to the principle of the colorimetry using a calibration curve created in advance. By keeping the amount of the aqueous liquid sample to be spotted, the incubation time, and the incubation temperature constant, the quantitative analysis of albumin can be performed with high accuracy. In the measurement operation, it is possible to perform a highly accurate quantitative analysis by a chemical analysis apparatus described in JP1985-125543A (JP-S60-125543A), JP1985-220862A (JP-S60-220862A), JP1986-294367A (JP-S61-294367A), JP1983-161867A (JP-S58-161867A), and the like in an extremely easy operation.

Next, the present invention will be described using Examples, but the present invention is not limited thereto.

### EXAMPLES

### Example 1

An aqueous solution having the following composition was applied to a 180 μm colorless transparent smooth film of polyethylene terephthalate, which had been subjected to a gelatin undercoating, and dried.

(Interlayer)

| | |
|---|---|
| Polymer AAm-VP-MAOH | 44.0 g/m² |
| Succinic acid | 3 g/m² |

The pH of the aqueous solution was set to 5.0.

The polymer AAm-VP-MAOH refers to a polyacrylamide-N-vinyl-2-pyrrolidone-methallyl alcohol ternary copolymer (monomer molar ratio of 57:38:5 (manufactured by FUJIFILM Corporation)) (hereinafter, the same applies).

Next, water was supplied to the entire surface of the above-described film in a supply amount of approximately 30 g/m² to wet the film, and then a tricot knitted fabric obtained by knitting a polyethylene terephthalate spun yarn corresponding to 50 denier at 36 gauge was laminated with light pressure, and dried.

Next, an ethanol (30% by mass) aqueous solution (pH 4.7) having the following composition was applied such that each component had the following amount, and dried to produce an integrated multilayer analytical element.

### (Reagent Holding Layer)

| | | |
|---|---|---|
| Succinic acid | 3.5 | g/m² |
| Polyvinylpyrrolidone | 8 | g/m² |
| BCP | 1.7 | g/m² |
| Surfactant Brij 35 | 0.3 | g/m² |
| Protein denaturant (component A) | 0.76 | g/m² |
| sodium dodecyl sulfate (SDS) | | |
| SH reagent (component B) | 0.1 | g/m² |
| 5,5′-dithiobis(2-nitrobenzoic acid) | | |

Here, as the surfactant, polyoxyethylene (23) lauryl ether (Brij 35, manufactured by FUJIFILM Wako Pure Chemical Corporation) which was a nonionic surfactant was used. The pH of the coating liquid used for forming the reagent holding layer was adjusted to 4.8 with succinic acid.

The above-described integrated multilayer analytical element was cut into a chip having a square of 12 mm×13 mm, and housed in a slide frame (described in JP1982-63452A (JP-S57-63452A)) to produce a test piece for measuring albumin according to the embodiment of the present invention (Example 1). At that time, the ratio of the content of the nonionic surfactant to the content of the bromocresol purple was 0.176.

### Examples 2 to 8

Test pieces for measuring albumin of Examples 2 to 8 were prepared in the same manner as in Example 1, except that the pH of the reagent holding layer was maintained as it was, and the type and amount of the nonionic surfactant and the coating amount of bromocresol purple (BCP) (component C) were changed such that the values in the table were obtained as shown in Table 1. In addition, the D/C ratio at that time was calculated and shown in Table 1. The protein denaturant (component A) and the SH reagent (component B) were used in the types and amounts shown in the table.

### Comparative Example 1

A test piece for measuring albumin was prepared by the same method as in Example 1, except that the amount of BCP in Example 1 was changed to 0.3 g/m². At that time, D/C was 1.0.

### Comparative Examples 2 to 7

As shown in the following table, the type and the amount of the reagent used were changed to prepare the test piece for measuring albumin.

### <Evaluation of Repeatability>

An examination liquid containing 4 g/dL of albumin (ALB) was prepared, and the repeatability was evaluated in a case where the measurement was performed 10 times. That is, the repeatability of the measured value indicates numerical variation in a case where the test piece for measuring albumin is repeatedly measured 10 times, and a smaller number means better performance.

The spotting amount was 10 μL. The results are shown in the following table.

The simultaneous repeatability is calculated as an average value (average: AV.) of 10 measurements, by calculating a sum of squares of differences between each measured value and an average value as standard sum of squares (SS).

The standard deviation was calculated as (SS/10)^{1/2}.

The coefficient of variation CV was calculated as CV=(standard deviation)/(AV.).

The simultaneous repeatability was represented by a CV value. A case where the CV value is low indicates that the simultaneous repeatability is good.

TABLE 1

| | Example 1 | Example 2 | Example 3 | Example 4 | Example 5 | Example 6 | Example 7 | Example 8 |
|---|---|---|---|---|---|---|---|---|
| Nonionic surfactants | Brij 35 | Tween 20 | Tween 80 | Triton | Emalgen | Brij 35 | Brij 35 | Emalgen |
| (component D) | | | | X100 | 120 | | | 120 |
| D/C ratio | 0.18 | 0.18 | 0.18 | 0.18 | 0.18 | 0.25 | 0.10 | 0.24 |
| Amount of BCP (C) | 1.7 | 1.7 | 1.7 | 1.7 | 1.7 | 0.8 | 2.8 | 2.4 |
| (g/m²) | | | | | | | | |
| Amount of nonionic | 0.3 | 0.3 | 0.3 | 0.3 | 0.3 | 0.2 | 0.28 | 0.58 |
| surfactant D (g/m²) | | | | | | | | |
| Amount of C (μg/10 | 265.2 | 265.2 | 265.2 | 265.2 | 265.2 | 109.2 | 78 | 78 |
| μL sample) in a case | | | | | | | | |
| of 10 μL spotting | | | | | | | | |
| Type of A | SDS | SDS | SDS | SDS | SDS | SDS | SDS | SDS |
| Amount (g/m²) | 0.76 | 0.76 | 0.76 | 0.76 | 0.76 | 0.86 | 0.96 | 0.26 |
| Type of B | DTNB | DTNB | DTNB | DTNB | DTNB | DTNB | DTNB | DTNB |
| Amount (g/m²) | 0.1 | 0.1 | 0.1 | 0.1 | 0.1 | 0.1 | 0.1 | 0.1 |
| Repeatability | 3.3% | 3.5% | 3.4% | 3.6% | 3.8% | 4.9% | 4.3% | 4.2% |

TABLE 2

| | Comparative | Comparative | Comparative | Comparative | Comparative | Comparative | Comparative |
|---|---|---|---|---|---|---|---|
| | Example 1 | Example 2 | Example 3 | Example 4 | Example 5 | Example 6 | Example 7 |
| Nonionic surfactants | Brij 35 | Brij 35 | Brij 35 | Brij 35 | Brij 35 | Brij 35 | Brij 35 |
| (component D) | | | | | | | |
| D/C ratio | 1 | 5 | 1 | 0.33 | 0.18 | 0.18 | 0.18 |
| Amount of BCP (C) | 0.3 | 0.06 | 0.06 | 1.5 | 1.7 | 1.7 | 1.7 |
| (g/m²) | | | | | | | |
| Amount of nonionic | 0.3 | 0.3 | 0.06 | 0.5 | 0.3 | 0.3 | 0.3 |
| surfactant D (g/m²) | | | | | | | |
| Amount of C (μg/10 | 46.8 | 9.4 | 9.4 | 46.8 | 265.2 | 265.2 | 265.2 |
| μL sample) in a case | | | | | | | |
| of 10 μL spotting | | | | | | | |
| Type of A | SDS | SDS | SDS | SDS | Absent | Absent | SDS |
| Amount (g/m²) | 0.76 | 0.76 | 0.76 | 0.76 | | | 0.01 |
| Type of B | DTNB | DTNB | DTNB | DTNB | Absent | DTNB | Absent |
| Amount (g/m²) | 0.1 | 0.1 | 0.1 | 0.1 | | 0.05 | |
| Repeatability | 10.8% | 9.8% | 16.5% | 10.5% | 6.5% | 6.2% | 6.4% |

As shown in the above table, the simultaneous repeatability (CV value) is good by satisfying that the reagent holding layer contains a protein denaturant and an SH reagent, the content of the bromocresol purple in the reagent holding layer is 0.78 g/m² to 3.81 g/m², and the ratio of the content of the nonionic surfactant to the content of the bromocresol purple is 0.01 to 0.3, whereby the effect of the present invention is confirmed.

What is claimed is:

1. A test piece for measuring albumin, comprising:
a support; and
a reagent holding layer provided over the support,
wherein the reagent holding layer contains a protein denaturant, an SH reagent, bromocresol purple, and a nonionic surfactant,
a content of the bromocresol purple in the reagent holding layer is 0.78 g/m² to 3.81 g/m², and
a ratio of a content of the nonionic surfactant to the content of the bromocresol purple is 0.01 to 0.3.

2. The test piece for measuring albumin according to claim 1,
wherein the content of the bromocresol purple in the reagent holding layer is 0.78 g/m² to 2.21 g/m², and
the ratio of the content of the nonionic surfactant to the content of the bromocresol purple is 0.01 to 0.2.

3. The test piece for measuring albumin according to claim 1,
wherein the reagent holding layer contains a water-soluble polymer.

4. The test piece for measuring albumin according to claim 3,
wherein the water-soluble polymer in the reagent holding layer is polyvinylpyrrolidone.

5. The test piece for measuring albumin according to claim 1, further comprising:
an interlayer between the support and the reagent holding layer.

6. The test piece for measuring albumin according to claim 5,
wherein the interlayer contains a water-soluble polymer, and
a content of the water-soluble polymer in the interlayer is 10 to 100 g/m².

7. The test piece for measuring albumin according to claim 1,
wherein the nonionic surfactant is a nonionic surfactant having a polyoxyethylene group.

8. The test piece for measuring albumin according to claim 1,
wherein the nonionic surfactant is one or more nonionic surfactants selected from the group consisting of polyoxyethylene lauryl ether, polyoxyethylene oleyl ether, polyoxyethylene sorbitan monolaurate, polyoxyethylene sorbitan monooleate, polyoxyethylene tert-octylphenyl ether, and polyoxyethylene isooctylphenyl ether.

9. The test piece for measuring albumin according to claim 1,
wherein the protein denaturant is sodium dodecyl sulfate.

10. The test piece for measuring albumin according to claim 1,
wherein the SH reagent is 5,5′-dithiobis(2-nitrobenzoic acid).

11. The test piece for measuring albumin according to claim 1,
wherein pH of a liquid of the reagent holding layer is 4.5 to 5.5.

12. A method for measuring albumin, comprising:
spotting an albumin-containing sample on the test piece for measuring albumin according to claim 1; and
measuring color development in the reagent holding layer.
